(19) United States
(12) Patent Application Publication
(10) Pub. No.: US 2013/0259928 A1
(43) Pub. Date: Oct. 3, 2013

(54) GENERATION OF VIROSOME PARTICLES

(71) Applicant: Maria Di Naro, Catania (IT)

(72) Inventor: Maria Di Naro, Catania (IT)

(73) Assignee: FRANVAX S.R.L., Catania (IT)

(21) Appl. No.: 13/876,793

(22) PCT Filed: Sep. 29, 2011
(86) PCT No.: PCT/EP2011/004874

§ 371 Date: Jun. 7, 2013

(30) Foreign Application Priority Data
Sep. 30, 2010 (EP) 10012574.9

(51) Int. Cl. A61K 9/127; A61K 39/145

(52) U.S. Cl. CPC A61K 9/1271; A61K 39/145; A61K 9/1277
USPC 424/450; 424/210.1

(57) ABSTRACT

The invention relates to the generation of a new class of virosome particles, making use of virus antigens expressed in plant, particularly influenza antigens, and to vaccines, particularly influenza vaccines, containing these virosome particles.

The invention relates to the generation of a new class of virosome particles, making use of virus antigens expressed in plant, particularly influenza antigens, and to vaccines, particularly influenza vaccines, containing these virosome particles.

### BACKGROUND OF THE INVENTION

Influenza, commonly known as flu, is one of the oldest and most common diseases. It is an acute respiratory illness characterized by different symptoms like fever, chills, cough, sore throat and headache. It is a very contagious disease transmitted by respiratory secretions through sneezing or coughing. Although it is most of the time a mild viral infection, influenza is responsible for high morbidity and mortality in infants, elderly and immunocompromised individuals (Cox N. J., Annu Rev Med, 2000, 51:4-7-421).

The vaccines against flu are based on the influenza virus surface protein (hemagglutinin, HA), which is the protective antigen. Current flu vaccines contain HA antigens from three different influenza strains, influenza A H1N1 and H3N2 and influenza B viruses. The emergence of new strains of seasonal influenza viruses as a result of antigenic drift requires the annual revision of the flu vaccine composition. Antigenic shift periodically (every 20 years on average) leads to pandemics, and currently the highly pathogenic H1N1 strain of the 2009 flu pandemic is of particular public health concerns.

Vaccination remains the most effective and cost-efficient way to prevent infection by influenza viruses in particular in the face of a threatening flu pandemic. Moreover, the worldwide capacity of seasonal flu vaccine production is limited to 400 million doses, which are far from meeting the 1 billion doses necessary to vaccinate high-risk individuals on a worldwide scale (Emmanuel E. J. and Wertheimer A., Science 2006, 312:854-855).

Antibodies to the influenza virus hemagglutinin (HA) play a major role in the protective ability of influenza vaccines. The molecule contains the binding site to target cell receptors and its variable globular domain expresses the majority of neutralization epitopes (Wiley D. C., Wilson I. A. and Skehel J. J., Nature 1981, 289:373-378). Commercial seasonal flu vaccines are based on inactivated or live attenuated flu viruses (Nichol K. L. and Treanor J. J., JID, 2006, 194 (Suppl.2), S111-S118). Subunit-based vaccine approaches in particular using baculovirus-expressed recombinant HA have been tested in clinical trials (Goji N. A., et al., JID, 2008, 1998:635-638).

Typically, it takes at least about 6 months to manufacture bulk quantities of new vaccines based on emerging viruses, which represents a significant hurdle to the development of a pandemic vaccine. In the case of the highly pathogenic H1N1 strain of the 2009 flu pandemic, the first cases were reported in Mexico in March 2009 (see WHO website), and the first corresponding vaccines, Focetria® (Novartis) and Pandemrix® (GlaxoSmithKline), were recommended for approval in Europe on Sep. 24, 2009 by EMEA (see EMEA website). Both were produced in hen eggs, and since the egg-based vaccine production apparently resulted in rather low titers, and correspondingly rather low immunogenicity of the vaccine, the addition of adjuvants was necessary in both cases.

Thus, the main production process today still involves an egg-based technique that cannot yield the number of vaccine doses that would be necessary to immunize all high-risk individuals worldwide. Generally, one egg is needed for the production of one dose of vaccine.

The process is faced with several limitations:
difficult and time-consuming logistics due to the high number of eggs needed;
limitation of production size and capabilities;
issue of production source in case of pandemic avian flu or pandemic 2009 flu;
sensitivity of the production process to contamination;
complexity and duration of the production process (6 months);
despite several purification steps, the vaccine might contain traces of avian proteins, which may cause undesirable allergic reactions in vaccinees.

In the case of the highly pathogenic H1N1 strain of the 2009 flu, the egg-based vaccine production additionally resulted in part in rather low titers, and correspondingly rather low immunogenicity of the vaccine, rendering the addition of adjuvants necessary.

Cell based technologies are beginning to compete with the egg-based process today. The most advanced technology is based on a canine kidney cell line called MDCK (Madin Darby Canine Kidney). It has some advantages as the logistic process is easier due to fact that the cells could be frozen and stored until the production process is started. The system is also less sensitive to contamination of the product and the vaccine itself does not contain residual traces of egg proteins that may potentially cause allergic reactions. Other cell-based technologies using Vero and PER.C6 cell cultures are under development. A vaccine against the highly pathogenic H1N1 strain of the 2009 flu, Celvapan® (Baxter; produced in Vero cells, not adjuvanted) was recommended for approval in Europe on Oct. 1, 2009 by EMEA (see EMEA website). However, regardless of the cell line used, these production processes have only limited capacity in terms of mass production. The rapid availability of massive quantities of an appropriate influenza antigen, however, represents a must to face the threat of influenza epidemics or pandemics.

Green biotech offers an opportunity to overcome the quantity problems related to current influenza vaccine production systems (eggs and mammalian cell culture). Another advantage of plants is that they are free of animal pathogens, making them safer production organisms for biopharmaceuticals.

However, also the production of influenza antigens in plants is not free of drawbacks. Contamination with plant material may lead to adverse allergic reactions and impede pharmaceutical approval. Therefore great care has to be taken when isolating and purifying the influenza antigens from plant extracts.

In another approach to enhance the potency of vaccines and thereby overcoming the availability problem, immunostimulating reconstituted influenza virosomes (IRIVs) were developed. IRIVs comprise an antigen or a combination of antigens incorporated into a virosome further containing a mixture of phospholipids, an essentially reconstituted functional virus envelope, and influenza hemagglutinin protein (HA) (cf. e.g. WO1992/19267).

Such IRIVs show for example very good results with antigens derived from inactivated Hepatitis A virus. However, in such IRIV vaccines no antibodies against HA were detected, indicating no immune response to the HA antigen, thus the use of “empty” IRIVs i.e. with influenza hemagglutinin protein alone, seemed not to be feasible as “stand alone” vaccines. Therefore, the prior art “empty” IRIVs were rather regarded as an adjuvant than a vaccine.

Recently a combination of green biotech and IRIV methods was published (WO 2009/009876; WO 2009/076778). In these experiments virus-like particles (VLPs) were produced in plants and isolated from the plant material. This new method allowed for a mass production of VLP particles. However, unfortunately, VLPs show a quite low immunogenicity making additional use of potent adjuvants necessary. Despite several approaches have been tested to enhance the efficacy of plant-derived influenza antigens, no plant-derived influenza vaccine has yet been approved.

Thus, the technical problem underlying the present invention was to provide new vaccines, particularly against influenza, which overcome the production limitations associated with the methods used in the state of the art, e.g. in terms of quantity, reproducibility and purity of the vaccines, while simultaneously maintaining immunogenicity of the vaccines.

A solution to the before-mentioned technical problems, i.e. vaccines, particularly influenza vaccines, that can be produced in high quantity with high reproducibility and purity, and simultaneously high immunogenicity, is neither provided nor suggested by the prior art.

The present invention solves the above technical problem by providing the embodiments characterized in the claims. By using these embodiments, it has become possible to increase the production capacities and quality of vaccines, particularly influenza vaccines.

The present invention may find applications in all fields of vaccines and vaccine production, particularly in influenza vaccines.

### SUMMARY OF THE INVENTION

The present invention provides a virosome particle comprising (i) a virus antigen produced recombinantly in plants and (ii) a lipid bilayer, wherein the lipid bilayer is characterized by at least one of the following features:
a) at least one bisacyloxypropylcysteine conjugate anchored in the lipid bilayer;
b) no phytosterols anchored in the lipid bilayer;
c) at least one zoosterol anchored in the lipid bilayer;
d) the same plasma membrane composition of the lipid bilayer as it is found in the plasma membrane of host cells for said virus;
e) no plant-derived sphingolipids anchored in the lipid bilayer.

In a particular embodiment, the invention relates to a synthetically produced virosome particle comprising (i) an influenza hemagglutinin (HA) antigen produced recombinantly in tobacco plants and (ii) a lipid bilayer, wherein the lipid bilayer comprises at least one bisacyloxypropylcysteine conjugate anchored in the lipid bilayer.

The present invention furthermore provides a vaccine containing a virosome particle according to the present invention, optionally in combination with a suitable pharmacologically acceptable substance diluent.

The present invention furthermore provides a method for producing a virosome particle comprising the steps of
a) producing a virus antigen recombinantly in plants;
b) producing a mixture of phospholipids, characterized by at least one of the following features:
   at least one bisacyloxypropylcysteine conjugate;
   no phytosterols;
   at least one zoosterol;
   the plasma membrane composition as it is found in the plasma membrane of host cells for said virus; and/or
   no sphingolipids;
c) reconstitution of the influenza virus antigen with said mixture of phospholipids to form said virosome particles.

The present invention furthermore provides a use of a virosome particle according to the present invention, a vaccine according to the present invention, or the virosome particle produced by the method according to the present invention for the prophylaxis of an infectious disease.

### FIGURES

The Figures show:

FIG. 1: Principle of the procedure of preparing virosome particles
(a) HA influenza antigen expressed in plant
(b) mixture of phospholipids
(c) solubilize influenza spike subunit antigens containing the HA with phospholipids in detergent
(d) virosome particle containing the reconstituted membrane carrying the influenza spike proteins including HA on the surface after detergent removal

FIG. 2: Silver stain of gradient

FIG. 3: Photon Correlation Spectroscopy (PCS)

FIG. 4: Immunogenicity of virosome particles in mice

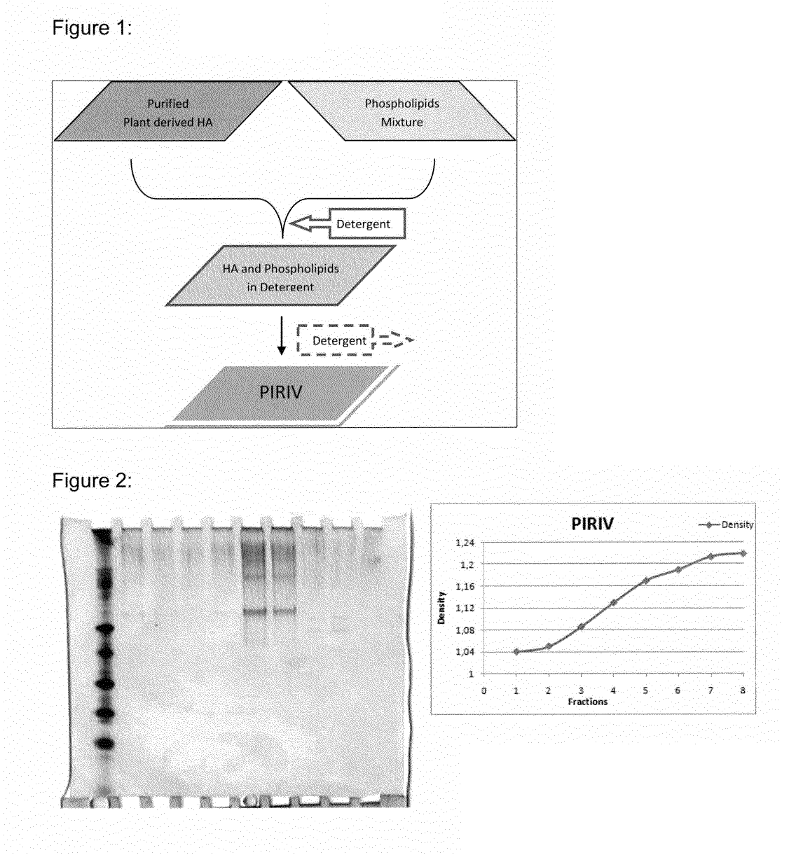

### DETAILED DESCRIPTION OF THE INVENTION

One aspect of the present invention relates to a virosome particle comprising (i) a virus antigen produced recombinantly in plants and (ii) a lipid bilayer, wherein the lipid bilayer is characterized by at least one of the following features:
a) at least one bisacyloxypropylcysteine conjugate anchored in the lipid bilayer;
b) no phytosterols anchored in the lipid bilayer;
c) at least one zoosterol anchored in the lipid bilayer;
d) the same plasma membrane composition of the lipid bilayer as it is found in the plasma membrane of host cells for said virus;
e) no plant-derived sphingolipids anchored in the lipid bilayer.

As used herein, the term “virosome particle” refers to a particle with a lipid bilayer containing a mixture of phospholipids, thus resembling an essentially reconstituted functional virus envelope. In a particular embodiment the lipid bilayer is in the form of a unilamellar bilayer.

As used herein, the term “virus antigen” may be any viral antigen that prompts the generation of antibodies and can cause an immune response.

In one embodiment such a viral antigen is an antigen derived from the family of Orthomyxoviridae. In particular such embodiments, the antigen is an influenza-derived antigen, in some embodiments of influenza A, B or C. In some embodiments, the antigen is selected from an influenza glycoprotein. In some embodiments the influenza antigen is selected from one or more members of the group consisting of hemagglutinin (HA), neuramimidase (NA), nucleoprotein (NP), M1-protein, M2-protein, NS1-protein, NS2(NEP)-protein, PA-protein, PB1-protein, PB1-F2-protein and PB2-protein. In particular embodiments, the virus antigen is hemagglutinin (HA). In further embodiments the influenza hemagglutinin is selected from the group consisting of H1, H2, H3, H4, H5, H6, H7, H8, H9, H10, H11, H12, H13, H14, H15 and H16, particularly H1.

In further embodiments, deletion, insertion or addition mutants (i.e. proteins with deleted, inserted or added amino acids or amino acid sequences) of such virus antigens are encompassed. Also chimeras (i.e. fusion proteins or protein-complexes of different origin), chemical modified proteins (e.g. pegylated proteins) and modified proteins (e.g. with additional, non-native amino acids) are encompassed. In one embodiment the plant derived antigen is derived from an influenza hemagglutinin. In further embodiments the virus antigen is derived from an influenza hemagglutinin selected from the group consisting of H1, H2, H3, H4, H5, H6, H7, H8, H9, H10, H11, H12, H13, H14, H15 and H16, particularly H1.

In particular embodiments, the viral antigen, e.g. hemagglutinin HA contains a trans-membrane region or derivative thereof.

In certain embodiments of the invention, the virus antigen is located in the lipid bilayer of the virosome particle.

In certain embodiments, the hemagglutinin (HA) is biologically active.

The term “biologically active” as used herein refers to HAs or derivatives which substantially display the full biological activity of native HA and are thus capable of mediating the adsorption of the virosome particles of the present invention to their target cells via sialic acid containing receptors. Furthermore, such HA components can be recognized by circulating anti-influenza antibodies. This biological activity is an essential feature of the virosome particles of the present invention

Without being bound to theory, the function of the HA component of the virosome particles of the present invention may be explained as follows:
1) it binds to a sialic acid (N-acetylneuraminic acid) containing receptor on a target cell to initiate the virosome particle-cell interaction;
2) it mediates the entry of the virosome particles into the cytoplasm by a membrane-fusion event and thus finally leads to its release; and
3) it serves as a “recognition antigen” since most humans can be considered “primed” to HA due to prior exposure through disease or vaccination.

Thus, the essential feature of such virosome particles is that they carry on their surface a biologically active viral glycoprotein (HA) or derivative thereof, avoiding an undesired long stay of the HA antigen in the endocytosomes, where it might be unspecifically degraded.

The fact that an antigen should be palatable for macrophages and other accessory cells is paramount. For this purpose, the particulate nature of the virosome particle is advantageous since it mimics the particulate entity of microorganisms.

Furthermore, since all human beings have antibodies against influenza antigen HA (either from a previous influenza infection or from a vaccination), antibody-antigen complexes (immune complexes) are rapidly formed. These immune complexes, however, accelerate the entry of recognized antigens not only into macrophages but also into lymphoid follicles, in which antigens are retained long-term in an extracellular location on the surface of follicular dendritic cells. Such a long-term extracellular presentation is of course a preferred feature of a vaccine due to its multifunctional immune-stimulatory effect (immunogenicity). This process of entering macrophages and lymphoid follicles is called opsonisation.

Furthermore, binding by antibody has another consequence for the immunogenicity of antigens. Whereas a given antigen, A, in solution will only bind to B cells exhibiting anti-body molecules of the specificity anti-A on their surface, immune complexes can adhere to any B cell via the Fc receptor. Due to the capacity of B cells in afferent lymph vessels to enter B cell areas of lymph nodes, this unspecific binding via the Fc receptor is probably one route, in a natural infection, by which said antigen is transported to lymphoid follicles and elsewhere in lymphatic tissue (Nossal, G.J.V., New Generation Vaccines (ed. Woodrow, G. C. and Levine, M. M.), Marcel Dekker, Inc., (1990) 85. The mechanism would be an adjunct to the transport by monocytes.

Hence, the presence of influenza antigens on the surface of the virosome particles favors the immunological mechanism of opsonisation.

In one embodiment, the virosome particles of the present invention contain the complete HA which is synthesized in plants as a single polypeptide chain of 550 amino acids which is subsequently cleaved by removal of arginine 329 (corresponding to arginine 345 of HA [Influenza A virus (A/TW/36/04(H3N2))], GenBank: ABD59855.1) into two chains HA1 (36,334 Daltons) and HA2 (25,750 Daltons).

These chains are optionally covalently linked by a disulfide bond involving the cysteine in HA1 position 14 and the cysteine in HA2 position 137 and the two-chain monomers are associated non-covalently to form trimers on the surface of IRIVs. These HA1 or HA2 peptides can be obtained from natural or synthetic sources or by genetic engineering.

Furthermore, the sudden application of large doses of pure protein antigens includes the risk of activating the suppressor pathways in the immune responses, particularly if the intravenous route is used; see Nossal, G.J.V., New Generation Vaccines, Marcel Dekker, Inc. New York, Basle (eds. Woodrow, Levine), (1990) 85. On the other hand a slow release permits extensive access of the antigen to the widely scattered dendritic cells and macrophages, and it also ensures that antigen will still be available after the initial burst of clonal proliferation, thereby permitting some facets of a secondary response. Thus the slow release of antigen as exhibited by virosome particles is another favorable feature for a vaccine.

As used herein, the term “produced recombinantly in plants” refers to the recombinant production of a protein, including a glycosylated protein, by expression in a plant host.

In the context of the present invention, the term “plant host” refers to any plant that is suitable for the recombinant expression of heterologous proteins.

In particular embodiments, the plant expression host is a tobacco plant, particularly Nicotiana bentamiana.

In certain embodiments of the invention, the virus antigen produced recombinantly in plants has a carbohydrate profile characteristic for the plant expression host.

Contrary to the present art techniques which produce whole virus-like particles (VLPs) in plants, only the virus antigen (e.g. the HA protein) is produced in plants and purified. Then the antigen is reconstituted with a mixture of phospholipids, which were not produced in plants.

As mentioned before, methods of the prior art normally produce the full VLPs in plants, i.e. the antigen as well as the phospholipid mixture and further proteins are of plant origin. The VLPs are then isolated from the plant products and formed by spontaneous aggregation. Although such a “one-step” procedure has the advantage of simplicity, it possesses several drawbacks.

First of all, it renders the production of contamination-free VLPs virtually impossible. Contamination with plant material, however, is a dangerous source for allergic or other adverse body reactions. Therefore, pharmaceutical approval is difficult for such VLP comprising vaccines.

Second, the spontaneous aggregation and particle formation renders any further process control impossible. This results in particles, which are inhomogeneous in size and composition.

Third, it is impossible to co-formulate adjuvants to the effect that they become part of the VLP. Instead, adjuvants can only be added after the particle formation already took place.

The present invention overcomes all of these disadvantages.

First, of all it has the advantage that it results in very pure virosome particles, since only the virus antigen (e.g. the HA protein) is produced in plants, whereas the phospholipids and other components of the particles are produced by chemical or biochemical means from non-plant sources.

Surprisingly the pure virosome particles of the invention show an enhanced immunogenicity as compared to the prior art VLPs. Without being bound to theory it is hypothesized, that since the pure virosome particles do not contain plant glycolipids and resemble more the structure of “native” virosomes, important epitopes of the antigens (e.g. HA proteins) are not masked and therefore the virosome particles of the present invention can induce a more potent immune response.

Secondly, the controlled addition of phospholipids (and other components) to the virus antigen (e.g. the HA protein) allows a controlled particle composition. That means both the size of the particles as well as their composition can be exactly governed and adjusted to individual needs. Furthermore, the immunogenicity can be improved by finetuning the composition of the particle.

The term “mixture of phospholipids” as used herein comprises natural or synthetic phospholipids or a mixture thereof. At least it contains one or more compounds selected from the group of glycerophospholipids, such as phosphatidylcholine or phosphatidylethanolamine, and cholesterol, particularly phosphatidylcholine and/or phosphatidylethanolamine.

The controlled aggregation of the particles allows for the incorporation (i.e. embedding or anchoring) of adjuvants into the lipid bilayer of the particle itself. To enhance the immunogenicity, in some embodiments said lipid bilayer comprises at least one bisacyloxypropylcysteine conjugate, which are anchored in the lipid bilayer resulting in stable particles ready for vaccination. The advantage of incorporating a bisacyloxypropylcysteine conjugate into the lipid bilayer of the virosome particle is that the ideal proportion between virosome particle surface, antigen distribution and adjuvant distribution can be kept stable.

In the context of the present invention, the term “bisacyloxypropylcysteine conjugate” refers to molecules of general formula I

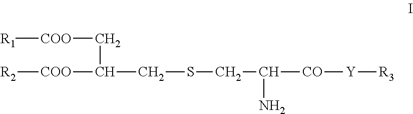

with
R₁ and R₂ being independently selected from alkyl or alkenyl groups, which form with the —C(═O)— group they are attached to an acyl group, such as palmitoyl;
Y being selected from —O—, —NH—, —S—, and —O—CO—, particularly —NH—; and
R₃ being a polymeric moiety suitable for incorporation into lipid bilayers, particularly a polypeptide, or a poly(ethylene glycol) moiety of general formula

—(CH₂—CH₂—O)ₘ—CH₂—CH₂—X

wherein
m is an integer selected from 5 to 700, particularly from 100 to 500; and
X is selected from —O—R⁴, —N(R⁴)₂, —S—R⁴, and —COOR⁴,
wherein R⁴ is selected from —H, -benzyl, C₁₋₆alkyl, and wherein in —N(R⁴)₂ the two residues R⁴ may be identical or different.

In additional embodiments, the virosome particles comprise a bisacyloxypropylcysteine conjugate according to formula II is envisaged:

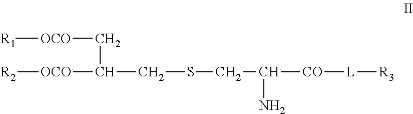

wherein
R₁ and R₂ can be identical or different and, together with the —OC-moiety they are attached to, for acyl moieties;
L is a linker moiety selected from the group of NH, O, S or OCO;
R₃ is a covalently linked conjugate moiety comprising at least two polyalkylene glycol units of the formula:

X₁—[(CHR₄)ₓ—O]ₙ—(CHR₄)_{y}—,

which may be identical or different;
where
—X₁ is hydrogen or a hydrocarbon, which may contain heteroatom(s);
R₄ is independently any one of hydrogen, OH, R₅OR₅ or CO—R₆;
R₅ is independently any one of hydrogen or C₁-C₆ alkyl;
R₆ is independently any one of hydrogen, OH, OR₅ or NR₇R₈;
R₇ and R₈ are independently any one of hydrogen or hydrocarbon which may contain heteroatom(s) and which may form a ring;
n is an integer of 1 to 100;
x is independently an integer of 1 to 10;
y is an integer of 0 to 10.

Therefore, in some embodiments of the present invention, the novel virosome particles comprise at least one bisacyloxypropylcysteine conjugate selected from the group comprising MALP-2 (see, for example, WO 98/27110 and WO 2003/084568), pegylated bisacyloxypropylcysteine (see, for example, WO 2004/009125), 4-ARM-bisacyloxypropylcysteine (particularly BPP-Glyc-Cys-4-arm-PEG; see, for example, WO 2007/059931)

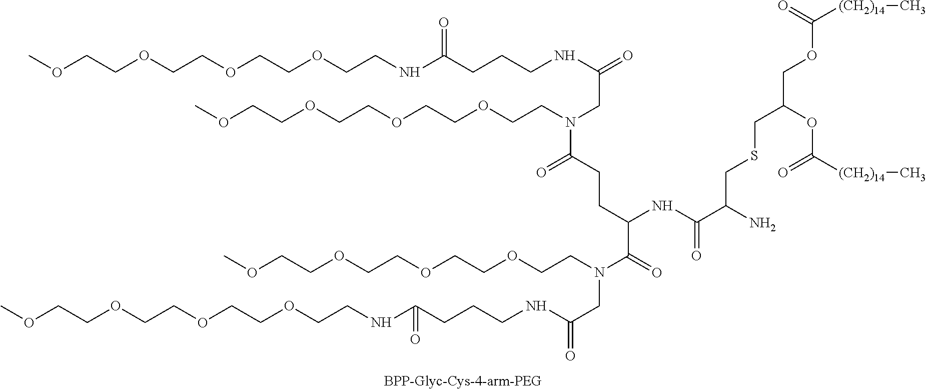

and other bisacyloxypropylcysteine conjugates, particularly MALP-2 and S—[2,3-bis(acyloxy)-(2R)-propyl]-L-cysteinyl-carboxy polyethylene glycol, particularly S—[2,3-bis(palmitoyloxy)-(2R)-propyl]-L-cysteinyl-carboxy polyethylene glycol.

The MALP-2 molecule and bisaxcyloxypropylcysteine conjugates thereof, e.g. a bispalmitoyloxypropylcysteine-PEG molecule, are known to represent potent stimulants for macrophages. The usefulness of MALP-2 as an adjuvant was shown previously, see e.g. WO 98/27110 and WO 2003/084568. The usefulness of a bispalmitoyloxypropylcysteine-PEG molecule as an adjuvant was shown previously, see e.g. WO 2004/009125. In particular, it was demonstrated that MALP-2 and bispalmitoyloxypropylcysteine-PEG molecules can act as an effective mucosal adjuvant enhancing the mucosal immune response, e.g. fostering an enhanced expression of antigen-specific IgA antibodies. Furthermore, it was shown that MALP-2 can activate dendritic cells and B-cells, both play an important role in the induction of a specific humoral immune response.

Therefore, in one embodiment, the virosome particles are for intranasal administration.

The term “phytosterols” refers to plant-derived sterols. There is some evidence that phytosterols can promote atherosclerosis, particularly in susceptible individuals. Therefore, in further embodiments, said lipid bilayer comprises no phytosterols. The lipid bilayer of the virosome particles of the present invention is especially free of campesterol, sitosterol and stigmasterol.

The term “zoosterol” refers to animal derived sterols, e.g. cholesterol. Cholesterol is an essential component of mammalian cell membranes, where it is required to establish proper membrane permeability and fluidity. Therefore, in another embodiment, the lipid bilayer may comprise at least one zoosterol, e.g. cholesterol.

As used herein, the term “the same plasma membrane composition of the lipid bilayer as it is found in the plasma membrane of host cells for said virus” refers to the fact that different kingdoms (Animalia, Plantae, Fungi, Protista, Archaea, Bacteria) differ in their composition of plasma membranes. Furthermore, indications exist that protein function as well as immune recognition of certain epitopes might be influenced by the specific lipid bilayer composition, since the physical properties of lipid bilayers (i.e. fluidity, polarity, permeability, stability etc.) depend to a great extend on their composition. Therefore, in some embodiments the specific lipid bilayer composition of the virosome particles of the invention resembles the composition of an animal or human lipid bilayer. In some embodiments the membrane-composition is similar to or the same as the membrane-composition of native influenza virosomes.

As used herein, the term “sphingolipids” refers to a class of lipids derived from the aliphatic amino alcohol sphingosine, including glycosphingolipids. These compounds play important roles in signal transmission and cell recognition. Plant-derived sphingolipids are major components of the plasma membrane, tonoplast, and other endomembranes of plant cells. To eliminate undesired cross-reactions between plant-derived sphingolipids and host immune system the lipid bilayer in some embodiments comprises no plant-derived sphingolipids.

Certain complex mammalian glycosphingolipids were found to be involved in specific functions, such as cell recognition and signaling. Said signaling involves specific interactions of the glycan structures of glycosphingolipids with similar lipids present on neighboring cells or with proteins. Thus, in some embodiments certain mammalian sphingolipids might be present in the virosome particles of the invention.

Other mammalian sphingolipids are commonly believed to protect the cell surface against harmful environmental factors by forming a mechanically stable and chemically resistant outer leaflet of the plasma membrane lipid bilayer. Such a “protective surface” however, reduces the chance of epitope-exposition to the host immune system, which is necessary for immunogenicity. Thus, in some embodiments the lipid bilayer of the virosome particles of the invention does not contain any sphingolipids at all.

The above mentioned features of the virosome particles of the present invention (i.e. containing bisacyloxypropylcysteine conjugates, not containing phytosterols, containing some zoosterols, having certain membrane-composition, and/or containing certain sphingolipids) might be combined by the person of ordinary skill in the art according to the situation at hand, the disease to be vaccinated and the antigen to be used. That is, for example an antigen of high immunogenicity might only be reconstituted in a virosome particle comprising a phosphatidylcholine lipid bilayer, whereas an antigen of low immunogenicity might be reconstituted together with immunogenicity enhancing substances like Bisacyloxypropylcysteine conjugates, zoosterols, or certain sphingolipids. In most embodiments no plant-derived material should be present in virosome particle of this invention, therefore phytosterols as well as plant-derived sphingolipids are to be avoided.

In certain embodiments of the invention, the virosome particle is produced synthetically.

Therefore, in a particular embodiment, the present invention relates to a synthetically produced virosome particle comprising (i) an influenza hemagglutinin (HA) antigen produced recombinantly in tobacco plants and (ii) a lipid bilayer, wherein the lipid bilayer comprises at least one bisacyloxypropylcysteine conjugate anchored in the lipid bilayer.

In certain embodiments, the virosome particles further comprises one or more additional adjuvants, including but not limited to lipopolysaccharides.

In the context of the present invention, the term “lipopolysaccharides” (or LPS), refers to molecules also known as lipoglycans, which are large molecules consisting of a lipid and a polysaccharide joined by a covalent bond; they are found in the outer membrane of Gram-negative bacteria, act as endotoxins and elicit strong immune responses in animals. Therefore, in some embodiments the lipid bilayer virosome particles may contain LPS as an additional immunostimulant.

LPS, as envisaged by this invention, comprises three parts:
1. O antigen (or O polysaccharide)
2. Core oligosaccharide
3. Lipid A

Lipid A is normally a phosphorylated glucosamine disaccharide decorated with multiple fatty acids. These hydrophobic fatty acid chains anchor the LPS into the bacterial membrane and the rest of the LPS projects from the cell surface. The lipid A domain is responsible for much of the toxicity of Gram-negative bacteria. When bacterial cells are lysed by the immune system, fragments of membrane containing lipid A are released into the circulation, causing fever, diarrhea, and possible fatal endotoxic shock (also called septic shock).

The core oligosaccharide attaches directly to lipid A and normally contains sugars such as heptose and 3-deoxy-D-mannooctulosonic acid (also known as KDO, ketodeoxyoctulosonate).

When LPS contains a repetitive glycan polymer this is referred to as the O antigen, O polysaccharide, or O chain of the bacteria. O antigen is attached to the core oligosaccharide, and comprises the outermost domain of the LPS molecule. The composition of the O chain varies from strain to strain, for example there are over 160 different O antigen structures produced by different E. coli strains. O antigen is exposed on the very outer surface of the bacterial cell, and as a consequence, is a target for recognition by host antibodies.

In an additional aspect of the invention, the invention relates to a vaccine containing a virosome particle according to the invention, optionally in combination with a suitable pharmacologically acceptable substance diluent.

The virosome particles of the present invention can be used as a potent active ingredient in an efficacious vaccine (e.g. influenza vaccine), which actively transport the desired antigen (e.g. HA protein) to APCs such as Macrophages, DC, B Cells, which will appropriately process and present said antigen to the immune system, as to induce a potent and protective immune response.

In a particular embodiment, the vaccine is in combination with a suitable pharmacologically acceptable substance adjuvant

In certain other such embodiments, the suitable pharmacologically acceptable substance adjuvant is co-formulated in the virosome particles.

In certain such embodiments, the suitable pharmacologically acceptable substance adjuvant is added to the virosome particles.

As used herein, the term “substance adjuvant” means substances which are coformulated and/or added in an immunization to the active antigen, i.e. the substance which provokes the desired immune response, in order to enhance or elicit or modulate the humoral and/or cell-mediated (cellular) immune response against the active antigen. Particularly, the adjuvant according to the present invention is also able to enhance or elicit the innate immune response.

To further enhance the immunogenicity of the new virosome particles, a large range of conventional adjuvants may be used. The most potent methods (e.g. administering the immunogen together with Freund's complete adjuvant) combine a number of separate principles explained in the following sections:

### (A) Chemical Immunopotentiation

A long history of research underlies the search for a pure, safe, effective, nontoxic small organic molecule which mimics the potentiation of the whole immune response as can be achieved with killed Mycobacterium tuberculosis bacteria or toxic microbial extracts, such as E. coli LPS.

(B) Co-Administration with Interleukins

There is some evidence that the co-administration of, for example, IL-2 with an antigen can result in a greater enhancement of the immune response than the separate administration of the antigen and the interleukin; see Staruch, M. J. and Wood, D. D., J. Immunol. 130 (1983), 2191.

(C) Co-Exhibition of the Antigens with a Highly Immunogenic Agent

If a particular vaccine is highly immunogenic, the adjuvant effect of this vaccine, and also the characteristics it may possess for guiding the response toward a particular immunological pathway, may “spill over” into a response to an antigen co-administered with it.

For example, killed Bordetella pertussis or Corynebacterium parvum bacteria are powerful immunogens. If a pure protein is administered with the same injection, the response to it is enhanced. Certain immunogens (for reasons that are unclear) guide the response in particular directions. For example, extracts of a parasite, such as Nippostrongylus brasiliensis, elicit powerful IgE responses. Pure proteins co-administered with the parasite extracts will also evoke an IgE response; see Nossal, G.J.V., New Generation Vaccines, Marcel Dekker, Inc. New York, Basle (eds. Woodrow, Levine), (1990) 85. Presumably, this effect is somehow connected to the production of particular lymphokines, which is induced by particular agents. Said lymphokines, such as IL-4, guide isotype switch patterns. The polyclonal activating characteristics of lymphokines may also form the basis for the enhancement of immune responses in general.

### (D) Hydrophobic Anchors and Immunostimulating Complexes

Surface-active agents such as saponin or Quil A in immunostimulating complexes (iscoms) have been used in a number of experimental and veterinary vaccines. They improved the immunogenicity of several antigens, especially of viral membrane proteins.

In particular embodiments, the substance adjuvant is selected from the list of bisacyloxypropylcysteine conjugates, and LPS.

In certain embodiments, the virosome particle-comprising vaccine is for intranasal administration.

Yet another aspect of the invention relates to a method for producing a virosome particle comprising the steps of.
a) producing a virus antigen recombinantly in plants;
b) producing a mixture of phospholipids, characterized by at least one of the following features:
   (I) at least one bisacyloxypropylcysteine conjugate;
   (ii) no phytosterols;
   (iii) at least one zoosterol;
   (iv) the plasma membrane composition as it is found in the plasma membrane of influenza host cells for said virus; and/or
   (v) no sphingolipids;
c) reconstitution of the influenza virus antigen with said mixture of phospholipids to form said virosome particles.

Yet another aspect, the invention relates to a use of a virosome particle of the present invention, a vaccine of the present invention or a virosome particle produced by the method of the present invention for the prophylaxis of an infectious disease.

In certain embodiments, the use of the present invention is for the prophylaxis of an infectious diseases comprising administering a suitable dosage of the virosome particles of the present invention, a vaccine of the present invention or a virosome particle produced by the method of the present invention to a patient in need thereof.

### EXAMPLES

The examples illustrate the invention.

### Example 1

### Preparation of Virosome Particles

Influenza haemagglutinin expressed and purified from Nicotiana bentamiana solubilized in PBS, is mixed with egg-derived lipids in powder (lecithins such as egg phosphatidylcholine) dissolved in PBS containing 100 mM OEG as detergent. The lipids protein ratio may vary from 20:1 to 1:10. In our hands the optimal ratio is 6:1. The lipids protein ratio can vary even more if other lipids (synthetic or steroid type) are used. Lipids and Influenza HA can be optionally submitted to ultrasound pulse. The mixture is then pass through a 0.22 mm filter and the detergent is removed through a series of different passages in SM-2 Bio-Beads. The detergent-removal drives the spontaneous assembly of the dissolved mixture of components in a population of virosome particles. After the last passage of SM-2 Bio-Beads the virosome particle population is submitted again to a 0.22 mm filtration and the final product is an homogenous virosome particle population with a mean size in diameter of 80-150 nm depending on the exact composition.

### Example 2

### Alternative Modification for Virosome Particle Generation

A lipid mixture such as egg derived phosphatidylcholine and phosphatidylethanolamine in powder are dissolved in PBS containing 100 mM OEG as detergent. The lipids ratio may vary from 20:1 to 1:10. In our hands the optimal range is 5:1. The lipids protein ratio may vary from 20:1 to 1:10. In our hands the optimal ratio is 7:1. The lipids protein ratio can vary even more if other lipids (synthetic or steroid type) are used or combination of different lipids are used. Lipids and Influenza HA can be optionally submitted to ultrasound pulse.

The solution is then mixed with Influenza haemagglutinin expressed and purified from Nicotniana bentamiana solubilized in PBS. Lipids and Influenza HA can be optionally submitted to ultrasound pulse. The mixture is then pass through a 0.22 mm filter and the detergent is removed through a series of different passages in SM-2 Bio-Beads. In the last step the detergent is removed by batch chromatography using SM-2 Bio-Beads. The removal drives the spontaneous assembly of components in homogeneous population of virosome particle with a mean diameter of 80-150 nm depending on the exact composition.

To solubilize lipids and protein the detergent of choice is OEG in PBS at a final concentration of 50 mM, however a concentration between 20 to 100 mM may be used. Detergents other than OEG, of non-ionic, ionic or zwitterionic nature may be used in the form

### Example 3

### Sucrose Gradient and Silver Staining

Sucrose gradient: An ultracentrifugation through a discontinuous sucrose gradient was applied as analytical method to assess antigen incorporation in virosome particles, based on the distinct densities of the individual components. Aliquots of virosome particle formulations in PBS were applied on the top of a 10-60% (w/v) discontinuous sucrose gradient in PBS and centrifuged at 100,000 g for 24 h at 4° C. The collected fractions were subsequently analyzed for density and followed by SDS PAGE and Silver staining to determine the fraction containing HA.

Silver staining: SDS Page was made according to supplier's instruction (Invitrogen). Silver Staining of gels was performed according to supplier's instructions (Bio-Rad)

### Example 4

### Particle Sizing: Photon Correlation Spectroscopy

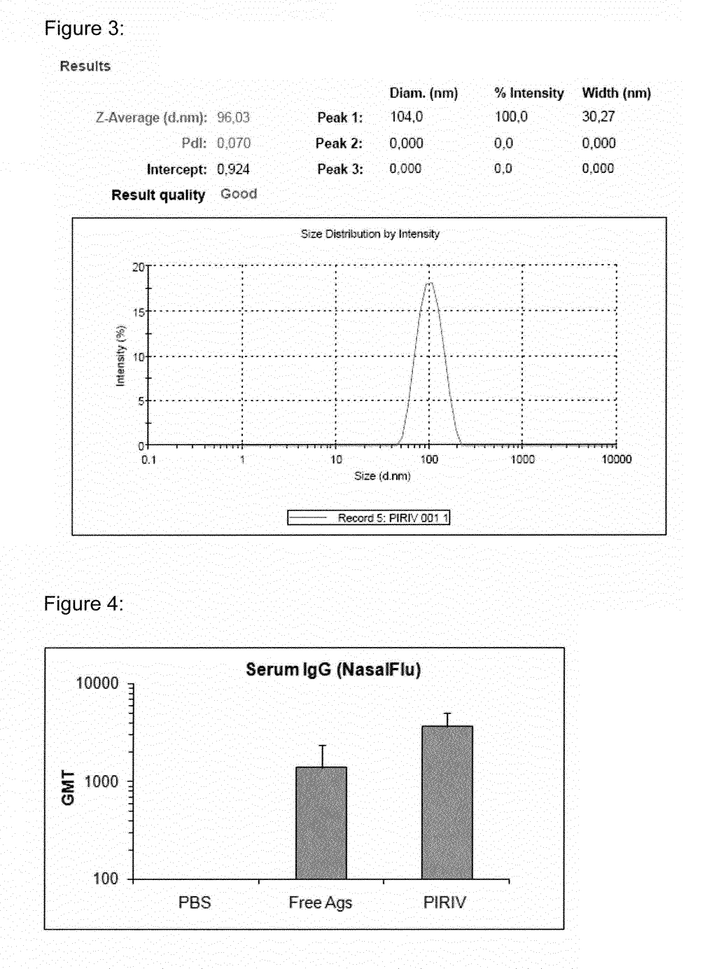

The hydrodynamic diameter, the polydispersity index, and the statistical particle size distribution of HA purified from a plant system as starting materials and formulated virosome particles were determined by Photon Correlation Spectroscopy or dynamic light scattering. This method relies on the size dependent speed of Brown's movements, which is measured as the variation of light scattering over time. A Malvern Zetasizer Nano (Malvern Ltd, Malvern, UK) was used for this purpose, including the software for the calculation of the parameters from the raw data, change of light intensity. The samples were diluted adequately in NaCl 0.9% for measurement, and 1 ml of the dilution was analyzed under standard conditions at 25° C. FIG. 3 shows the analysis of virosome particles generated according to example 1.

### Example 5

### Immunogenicity of Virosome Particles in Mice

Vaccine: Comparison of two different virosome particle formulations prepared according to Example 1 with free plant-derived HA antigen.

The immunogenicity of the formulation has been tested in a mice model. Experiments were performed using a virosome particle formulation in comparison with free antigens. Mice were immunized with two intramuscular injections at day 0 and day 7. Three weeks after the second immunization blood was withdrawn and analyzed for serum antibody. Results expressed as mean geometric titer are summarized in FIG. 4.

The numbers on the columns represent the range of the anti-HA antibody titer. The geometric mean titer (range) for the free antigen and virosome particle vaccine formulation on day 28 was 1393 (800-3200) and 3676 (3200-6400), respectively. Thus, the virosome particle preparations of the present invention are superior to free antigen vaccine.

1.-11. (canceled)

12. A synthetically produced virosome particle comprising (i) an influenza hemagglutinin (HA) antigen produced recombinantly in tobacco plants and (ii) a lipid bilayer, wherein the lipid bilayer comprises at least one bisacyloxypropylcysteine conjugate anchored in the lipid bilayer, and wherein said influenza hemagglutinin (HA) antigen is biologically active.

13. The virosome particle according to claim 12, wherein said influenza hemagglutinin (HA) antigen is located in the lipid bilayer of said virosome particle.

14. The virosome particle according to claim 12, wherein said influenza hemagglutinin (HA) antigen is located on an outer surface of said virosome particle.

15. The virosome particle according to claim 12, wherein the virosome particle is free of phytosterols.

16. The virosome particle according to claim 12, wherein the bisacyloxypropylcysteine conjugate is selected from the group consisting of MALP-2, a pegylated bisacyloxypropylcysteine, and a 4-ARM-bisacyloxypropylcysteine.

17. The virosome particle according to claim 16, wherein the pegylated bisacyloxypropylsysteine is S—[2,3-bis(palmitoyloxy)-(2R)-propyl]-L-cysteinyl-carboxy polyethylene glycol.

18. The virosome particle according to claim 16, wherein the 4-ARM-bisacyloxypropylcysteine is BPP-Glyc-Cys-4-arm-PEG.

19. A vaccine comprising a plurality of virosome particles according to claim 1 and optionally a pharmacologically acceptable substance diluent.

20. The vaccine according to claim 19, further comprising a suitable pharmacologically acceptable substance adjuvant.

21. The vaccine according to claim 19, wherein the vaccine is formulated for intranasal administration.

22. A method of producing virosome particles, comprising:
a) providing an influenza hemagglutinin (HA) antigen that has been recombinantly produced in plants;
b) providing a mixture of phospholipids, wherein the mixture of phospholipids comprises at least one bisacyloxypropylcysteine conjugate;
c) mixing together the influenza hemagglutinin (HA) antigen and the mixture of phospholipids; and
d) reconstituting the influenza hemagglutinin (HA) antigen with the mixture of phospholipids to form said virosome particles.

23. The method according to claim 22, further comprising filtering the mixture comprising the influenza hemagglutinin (HA) antigen, the mixture of phospholipids, and the detergent.

24. The method according to claim 22, wherein the ratio of phospholipids to influenza hemagglutinin (HA) antigen ranges from 20:1 to 1:10.

25. The method according to claim 22, further comprising sonicating the mixture comprising the influenza hemagglutinin (HA) antigen, the mixture of phospholipids, and the detergent.

26. The method according to claim 22, wherein the virosome particles have an average diameter within a range of 80 nm to 150 nm.

27. The method according to claim 22, wherein the influenza hemagglutinin (HA) antigen is recombinantly produced in Nicotiana benthamiana.

28. The method according to claim 22, wherein the influenza hemagglutinin (HA) antigen and the mixture of phospholipids are mixed together with a detergent to form a solubilized antigen-lipids mixture, and wherein reconstituting the influenza hemagglutinin (HA) antigen with the mixture of phospholipids to form said virosome particles comprises removing the detergent from the solubilized antigen-lipids mixture to spontaneously assemble said virosome particles.

29. A method of vaccinating or immunizing a patient against influenza, the method comprising administering to the patient a suitable dosage of the virosome particle according to claim 12.
